(19) United States
(12) Patent Application Publication
(10) Pub. No.: US 2022/0096764 A1
(43) Pub. Date: Mar. 31, 2022

(54) SYNCHRONIZED HIGH-FLOW SYSTEM

(71) Applicant: Covidien LP, Mansfield, MA (US)

(72) Inventors: Kun Li, San Diego, CA (US); Cynthia A. Miller, Laguna Hills, CA (US)

(73) Assignee: Covidien LP, Mansfield, MA (US)

(21) Appl. No.: 17/366,330

(22) Filed: Jul. 2, 2021

Related U.S. Application Data
(60) Provisional application No. 63/083,600, filed on Sep. 25, 2020.

(51) Int. Cl. A61M 16/00; A61M 16/06; A61M 16/20

(52) U.S. Cl. CPC A61M 16/0003; A61M 16/0672; A61M 16/201; A61M 2016/003; A61M 2205/50; A61M 2016/0027; A61M 2205/70

(57) ABSTRACT

Systems and methods for a synchronized high-flow mode are disclosed. In examples, the synchronized high-flow mode varies the flow of breathing gases delivered to the patient to account of changes in a patient's peak inspiratory flow demand, improve the accuracy of measured partial pressure of CO₂, maintain a more consistent distending pressure, reduce entrainment of room air, and enhance patient synchronization. In an example, a synchronized high-flow mode provides a baseline flow and increases the delivered flow above the baseline flow when the patient's demand exceeds a threshold. In this way, a synchronized high-flow mode may deliver a variable, or time-varying, flow based on the patient demand. Additionally, a minimum baseline flow may be selected and adjusted to maintain a minimum PEEP level.

### CROSS-REFERENCE TO RELATED APPLICATIONS

This application claims the benefit of U.S. Provisional Application No. 63/083,600, filed Sep. 25, 2020, the complete disclosure of which is hereby incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, modern ventilators may be customized for the particular needs of an individual patient. For example, several different ventilator modes or settings have been created to provide better ventilation for patients in different scenarios, such as mandatory ventilation modes, spontaneous ventilation modes, and mixed ventilation modes (e.g., SIMV, BiLevel). Ventilators monitor a variety of patient parameters and are well-equipped to provide reports and other information regarding a patient's condition.

It is with respect to this general technical environment that aspects of the present technology disclosed herein have been contemplated. Furthermore, although a general environment is discussed, it should be understood that the examples described should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for a synchronized high-flow oxygen therapy system (otherwise referred to herein as a synchronized high-flow system that may operate in a synchronized high-flow mode). Aspects disclosed herein include a method for synchronizing flow delivered to a patient at a patient interface. The method includes determining a resistance of a breathing circuit including a patient interface and delivering a baseline flow of breathing gases at a flow valve upstream from the patient interface. The method further includes measuring an upstream flow and an upstream pressure, the upstream flow and the upstream pressure determined at a position between the flow valve and the patient interface. Additionally, the method includes estimating an interface pressure at the patient interface. Based on the interface pressure, the method includes determining a delta flow. The method also includes delivering an augmented flow at the flow valve, wherein the augmented flow equals the baseline flow plus the delta flow.

In an example, the baseline flow is delivered according to a synchronized high-flow mode. In another example, determining the resistance includes performing a calibration procedure by varying the upstream flow and the upstream pressure. In a further example, estimating the interface pressure is based on the upstream flow, the upstream pressure, and the resistance. In yet another example, the patient interface is a nasal cannula and the interface pressure is estimated at nares of the nasal cannula. In still a further example, determining the delta flow is further based on a pressure difference between the interface pressure and a baseline pressure. In another example, the delta flow is equal to a non-negative value proportional to the pressure difference. In a further example, the baseline pressure is zero. In yet another example, the delta flow is time-varying and non-negative.

In another aspect, a method for synchronizing flow delivered to a patient at a patient interface during a synchronized high-flow mode is disclosed. The method includes delivering a constant baseline flow of breathing gases at a flow valve upstream from a patient interface. Additionally, the method includes measuring an upstream flow and an upstream pressure, wherein the upstream flow and upstream pressure are determined at a position between the flow valve and the patient interface. Based on the upstream flow and the upstream pressure, the method includes estimating a time-varying interface pressure. Additionally, the method includes determining that the time-varying interface pressure at the patient interface is less than a baseline pressure. Based on the time-varying interface pressure and the baseline pressure, the method includes determining a time-varying delta flow. The method further includes delivering, at the flow valve, the time-varying delta flow in addition to the constant baseline flow.

In an example, the patient interface is a nasal cannula and the time-varying interface pressure is determined at nares of the nasal cannula. In another example, the method further includes determining a resistance for a breathing circuit including the patient interface. In a further example, estimating the time-varying interface pressure is further based on the resistance. In yet another example, the resistance is determined according to a calibration procedure that varies the upstream flow and the upstream pressure. In still a further example, the time-varying delta flow is further based on a pressure difference between the time-varying interface pressure and the baseline pressure. In another example, the time-varying delta flow is equal to zero, when the pressure difference is less than a threshold. In a further example, the baseline flow is at least 2 liters per minute.

In a further aspect, a ventilator is disclosed. The ventilator is capable of synchronizing flow delivered to a patient at a patient interface. The ventilator includes a flow valve fluidly coupled to a breathing circuit including a patient interface. The ventilator further includes a flow sensor positioned downstream from the flow valve and upstream from the patient interface. Additionally, the ventilator includes a pressure sensor positioned downstream from the flow valve and upstream from the patient interface. The ventilator also includes a processor and memory storing instructions that, when executed by the processor, cause the ventilator to perform a set of operations. The set of operations include determining a resistance for the breathing circuit including the patient interface and delivering a baseline flow at the flow valve. The set of operations further includes measuring an upstream flow at the flow sensor and an upstream pressure at the pressure sensor. Based on the upstream flow and the upstream pressure, the set of operations includes estimating an interface pressure at the patient interface. Based on the interface pressure, the set of operations includes determining a delta flow. Additionally, the set of operations includes delivering an augmented flow through the patient interface, wherein the augmented flow equals the baseline flow plus the delta flow.

In an example, the delta flow is proportional to a pressure difference between the interface pressure and the baseline pressure when the pressure difference is at least a threshold. In another example, the delta flow is time-varying and non-negative. In a further example, determining the resistance includes performing a calibration procedure by varying the upstream flow and the upstream pressure.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating an example of a medical ventilator connected to a human patient.

FIG. 2 is a block diagram illustrating an example of a ventilator system.

FIG. 3 is a circuit diagram illustrating the flow of breathing gas through a ventilator to be delivered to the patient nares.

FIG. 4A shows an example method of a calibration process for a breathing circuit including a patient interface.

FIG. 4B shows an example of a graphical representation of the calibration process of FIG. 4A.

FIG. 5 is a block diagram illustrating an example system for implementing a synchronized high-flow mode using a high-flow system.

FIG. 6 shows a graphical example of an implemented synchronized high-flow mode.

FIGS. 7A-B show example methods of a synchronized high-flow mode.

FIGS. 8, 9, and 10 are additional example methods of a synchronized high-flow mode.

While examples of the disclosure are amenable to various modifications specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, gas cylinders, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

For the purposes of this disclosure, a “breath” refers to a single cycle of inhalation and exhalation delivered with the assistance of a ventilator. The term “breath type” refers to some specific definition or set of rules dictating how the pressure and flow of respiratory gas is controlled by the ventilator during a breath.

Owing to its potential of reducing lung injury and ventilator associated events associated with mechanical ventilation, the use of noninvasive respiratory support, particularly nasal continuous positive airway pressure (CPAP), has become a common strategy for early respiratory management of preterm infants. In recent years, high flow oxygen therapy systems (HFO₂T systems or high-flow systems) have increased in popularity as an alternative form of non-invasive respiratory support. An example high-flow system with increased popularity for respiratory support of infants is a heated, humidified, high-flow nasal cannula system (HFNC system). High-flow systems may generally include a ventilator or a device for controlling breath delivery, a humidifier, ventilation tubing, and a patient interface (e.g., nasal cannula, tracheostomy mask, endotracheal tube tee piece, face mask, nasal mask, etc.) compatible with delivery of high flow to the patient. In contrast to nasal CPAP, for which the rationale is essentially based on the provision of a continuous distending pressure, multiple mechanisms have been suggested to explain high-flow system functions, such as washout of the nasopharyngeal dead space, optimal gas conditioning, and provision of a variable distending pressure. Compared to nasal CPAP systems, a high-flow system may offer ease of use, reduced risk of nasal injuries, better infant tolerance with improved feeding, and bonding. Another benefit of high-flow systems is generating positive end expiratory pressure (PEEP). The appropriate level of PEEP can reduce work of breathing. The clinicians may adjust the flow rate in the high-flow system to achieve a desired PEEP level.

High-flow systems have traditionally used a constant flow rate that does not change. The value for that constant flow rate used for high-flow systems varies across published trials. This variability in the value for the constant flow rate reflects uncertainty about which flow rate, or range of flow rates, is likely to be effective in preterm infants and what factors might determine pressure transmission to infants. In one study, it was shown that setting the flow rate through the patient interface of a HFNC to 1 L/min/kg only meets an infant's peak inspiratory flow (PIF) in 78% of patients. In contrast, a flow rate of 2 L/min/kg was shown to always exceed an infant's PIF. The PIF may vary from patient to patient. If the flow rate through the patient interface is less than a patient's PIF, the patient may not receive the intended benefits of high-flow systems. Conversely if the flow setting exceeds the patient's PIF, the patient may experience adverse effects such as lung distention and/or air leaks.

Additionally, if the flow rate is too high, measurements of the partial pressure of CO₂ (P_{CO2}) of the patient may be impacted (e.g., partial pressure of carbon dioxide in arterial blood, Pa_{CO2}, end-tidal partial pressure of carbon dioxide, Et_{CO2} or Pet_{CO2}, mixed exhaled P_{CO2}, P_{ĒCO2}, measured mixed exhaled PCO2 including gas compressed in the ventilator circuit, Pexh_{CO2}, transcutaneous P_{CO2}, Pt_{CCO2}, mixed venous P_{CO2}, or any other partial pressure of carbon dioxide). Partial pressure of CO₂, P_{CO2}, monitoring has becoming a standard practice in NICU and PICU to determine how well the patient is clearing flow from the airway. A monitor may be placed in the dead space between the patient nares and the patient interface to measure P_{CO2}. If the flow through the patient interface is too high, the measured P_{CO2} is diluted by the excess flow and causes the P_{CO2} to be artificially low. Thus, with high-flow systems, if the flow rate is too high, the accuracy of P_{CO2} is inevitably impacted. Merely reducing the flow rate through the high-flow patient interface, such as to attain a PEEP level, however, may still not meet the patient's PIF.

The systems and methods disclosed herein address these circumstances, among other things, by providing a synchronized high-flow mode that may be implemented by a ventilator using a high-flow system. The synchronized high-flow mode varies the flow of breathing gases delivered to the patient to allow for a patient's PIF demand to be more accurately met while providing additional improvements. In an example, a synchronized high-flow mode provides a baseline flow and increases the delivered flow above the baseline flow when the patient's demand exceeds a threshold. In this way, a synchronized high-flow mode may deliver a variable peak flow based on the patient demand. Clinicians may still provide or adjust at least a baseline flow to maintain a PEEP level to the patient. The baseline flow (relative to that delivered at the patient's PIF) may improve the accuracy of measurements for partial pressure of CO₂, while patient's peak flow demand is still met. Other benefits of a synchronized high-flow mode may include more consistent distending pressure, a variable flow rate to match each patient's PIF demand from breath to breath, a reduction in room air entrainment, and better patient synchronization. With these concepts in mind, several examples of synchronized high-flow methods and systems are discussed below.

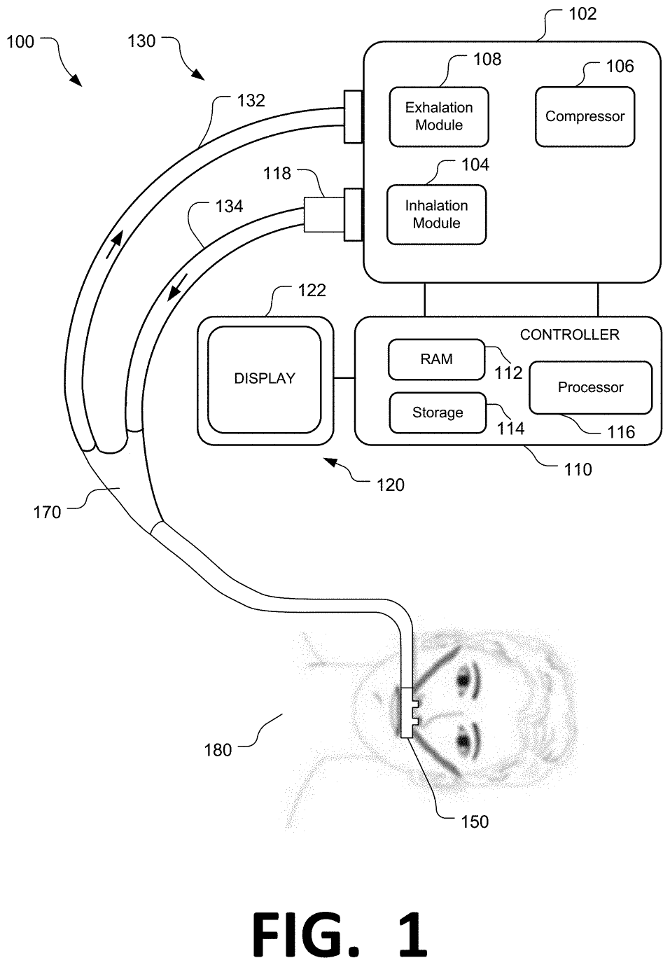

FIG. 1 is a diagram illustrating an example of a medical ventilator 100 connected to a patient 150. The ventilator 100 may provide positive pressure ventilation to the patient 150. Ventilator 100 includes a pneumatic system 102 (also referred to as a pressure generating system 102) for circulating breathing gases to and from patient 150 via the ventilation tubing system 130. The ventilation tubing couples the patient 150 to the pneumatic system via a patient interface 180. The breathing circuit includes the ventilation tubing system 130 and the patient interface 180. The patient interface 180 may be invasive (e.g., endotracheal tube, as shown) or non-invasive (e.g., nasal mask, nasal cannula). In an example, a noninvasive nasal interface includes two nasal prongs sized to fit inside the nostrils (the nasal nares) of the patient 150, however, other non-invasive interfaces may be supported (e.g., mask). The ventilator 100 controls the flow of gases into the ventilation tubing system 130 by controlling (adjusting, opening, or closing) an inhalation flow valve which may be part of the inhalation module 104. Additionally, a humidifier 118 may be placed along the ventilation tubing system 130 to humidify the breathing gases being delivered to the patient 150. A pressure sensor and flow sensor may be located at or near the inhalation module 104 and/or the exhalation module 108 to measure flow and pressure.

The ventilation tubing system 130 may be a two-limb circuit (shown) or a one-limb circuit for delivering breathing gases to the patient 150. In a two-limb example, a fitting, typically referred to as a “wye-fitting” 170, may be provided to couple a patient interface 180 to an inhalation limb 134 and an exhalation limb 132 of the ventilation tubing system 130. In a one-limb circuit (otherwise referred to herein as a single limb circuit), there may not be a wye fitting 170, an exhalation limb 132, or an exhalation module 108, or the breathing gases may be otherwise prevented from entering the exhalation limb 132.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator).

Controller 110 is operatively coupled with pneumatic system 102, signal measurement and acquisition systems, and an operator interface 120 that may enable an operator to interact with the ventilator 100 (e.g., change ventilation settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilator system or between the ventilator system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

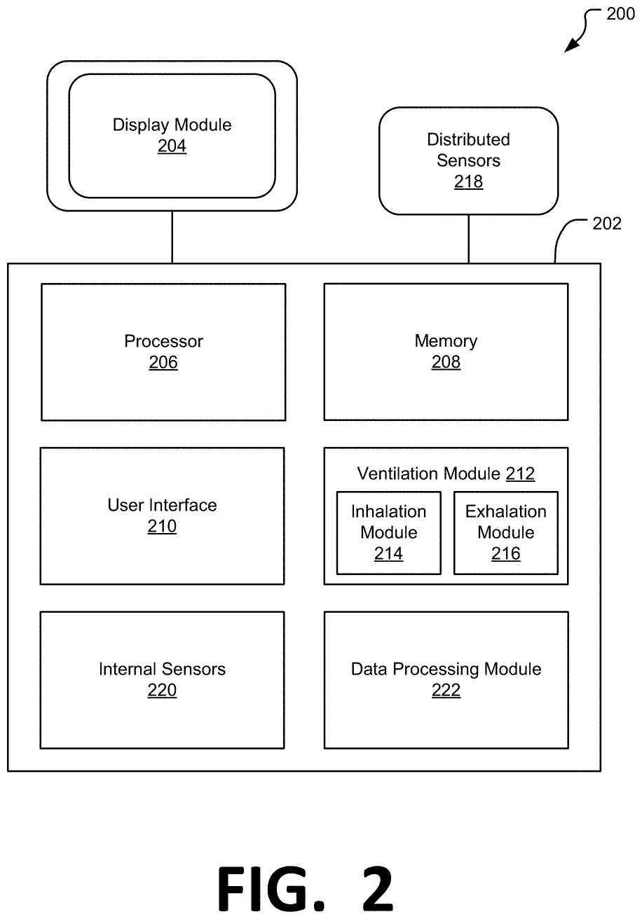

FIG. 2 is a block-diagram illustrating an example of a ventilator system 200. Ventilator system 200 includes ventilator 202 with various modules and components. That is, ventilator 202 may further include, among other things, memory 208, one or more processors 206, user interface 210, and ventilation module 212 (which may further include an inhalation module 214 and an exhalation module 216). Memory 208 is defined as described above for ventilation module 212. Similarly, the one or more processors 206 are defined as described above for one or more processors 206. Processors 206 may further be configured with a clock whereby elapsed time may be monitored by the ventilator system 200.

The ventilator system 200 may also include a display module 204 communicatively coupled to ventilator 202. Display module 204 provides various input screens, for receiving input, and various display screens, for presenting useful information. Inputs may be received from a clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 202 may be provided, for instance by a wheel, keyboard, mouse, or other suitable interactive device. Thus, user interface 210 may accept commands and input through display module 204, such as a baseline flow, PEEP, a high-flow mode, or other parameters related to a synchronized high-flow mode. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed in the form of graphs, wave representations (e.g., a waveform), pie graphs, numbers, or other suitable forms of graphic display. For example, the data processing module 222 may be operative to determine a ventilation settings (otherwise referred to as ventilatory settings, or ventilator settings, or ventilation settings) associated with a synchronized high-flow mode, display information regarding the synchronized high-flow mode, or may otherwise use the synchronized high-flow mode in connection with the ventilator, as detailed herein.

Ventilation module 212 may control ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with a synchronized high-flow mode. In some cases, certain ventilation settings may be adjusted based on the exhalation flow, e.g., to adjust or improve the prescribed treatment. Ventilation settings may include inhalation flow, frequency of delivered breaths (e.g., respiratory rate, (f), tidal volume (V_{T}), PEEP level, etc.).

Ventilation module 212 may further include an inhalation module 214 configured to deliver gases to the patient and an exhalation module 216 configured to receive exhalation gases from the patient, according to ventilation settings that may be based on the exhalation flow. As described herein, inhalation module 214 may correspond to the inhalation module 104, or may be otherwise coupled to source(s) of pressurized gases (e.g., air, oxygen, and/or helium), and may deliver gases to the patient. As further described herein, exhalation module 216 may correspond to the exhalation module 108, or may be otherwise coupled to gases existing the breathing circuit.

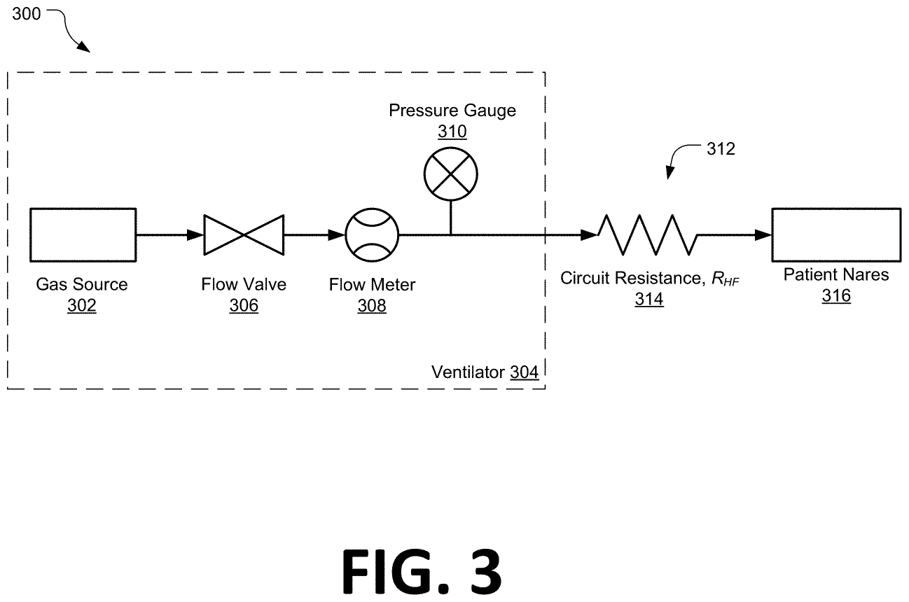

FIG. 3 is a circuit diagram 300 illustrating the flow of breathing gas from a gas source 302 through a ventilator 304 to be delivered to a patient at patient nares 316 in a high-flow system to be used in connection with a synchronized high-flow mode (e.g., in the case where the patient interface of the high-flow system interfaces with the patient nares 316). Other interfaces (as may or may not interface with the patient nares 316) with different associated circuit resistances R_{HF} should be appreciated. As shown, a gas source 302 may be coupled to, or otherwise delivered to, the ventilator 304. Ventilator 304 may have one or more components described with respect to the ventilators 100, 202 shown in FIGS. 1-2. As shown, the ventilator 304 includes a flow valve 306 to control the flow of breathing gases from the gas source 302 into the inhalation limb (such as inhalation limb 134) of the breathing circuit 312. The gas source 302 may be a mixing module, accumulator, or other ventilator component containing mixed gases at a desired concentration of oxygen to be delivered to the patient as breathing gases. The flow valve 306 is controlled by the ventilator 304 and may be associated with an inhalation module of the ventilator (such as inhalation modules 104, 214).

As the breathing gas (originating from the gas source 302) passes through the flow valve 306, the flow rate and pressure of the breathing gas may be measured by the ventilator using one or more sensors. The sensors may be positioned inside the ventilator or near the inhalation module of the ventilator 304 so as to take measurements “upstream” of the breathing circuit 312. The breathing circuit 312 includes a ventilation tubing system (such as ventilation tubing system 130) and the patient interface (such as patient interface 180). As shown, the upstream flow rate, Qᵢ, of the breathing gas is measured at a flow meter 308 and the upstream pressure, Pᵢ, of the breathing gas is measured at a pressure gauge 310. Although the flow meter 308 is shown before the pressure gauge 310 along the circuit diagram 300, it should be appreciated that the flow meter 308 and pressure gauge 310 may be positioned along the circuit diagram in any order, but are both positioned upstream of the patient interface of the breathing circuit 312. The flow meter 308 may be positioned upstream or downstream of the flow valve 306. The flow meter 308 and/or the pressure gauge 310 may be inside or outside of the ventilator 304.

After passing through the flow meter 308 and the pressure gauge 310, the breathing gas enters the breathing circuit 312. In the breathing circuit 312, the breathing gas flows through ventilation tubing of the inhalation limb and into a patient interface to the patient nares 316. The breathing circuit 312 is associated with a circuit resistance, R_{HF}, 314. The circuit resistance is based on the type of ventilation tubing, the length of ventilation tubing, and the type of patient interface of the circuit. The values for the circuit resistance may change based on the inhalation flow, Qᵢ, measured at the flow meter 308. In the example shown, the breathing gas is delivered to the patient at the patient nares 316, as may be attained using a non-invasive patient interface (e.g., nasal mask or nasal cannula).

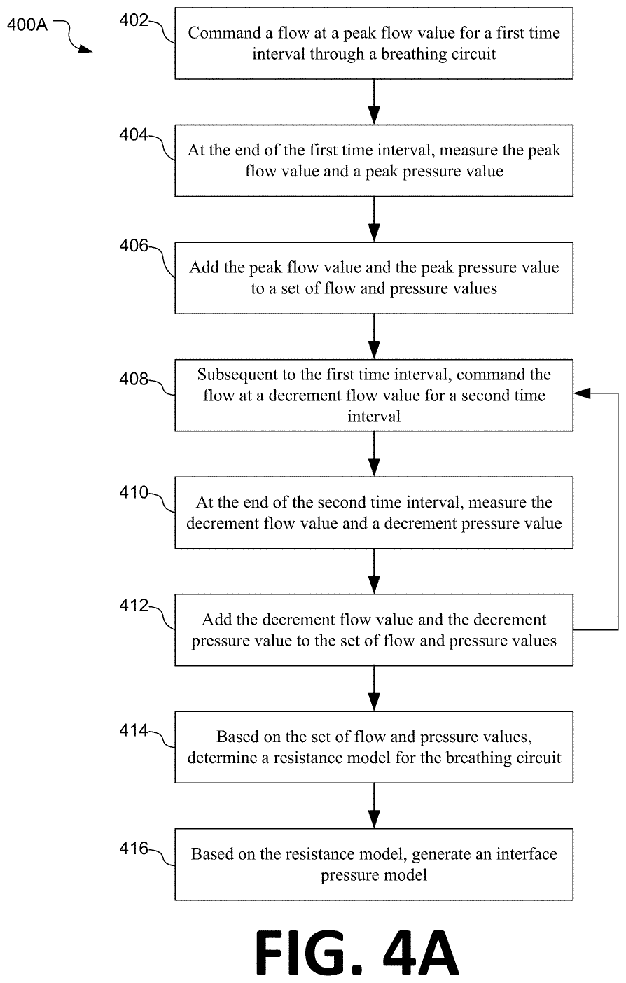

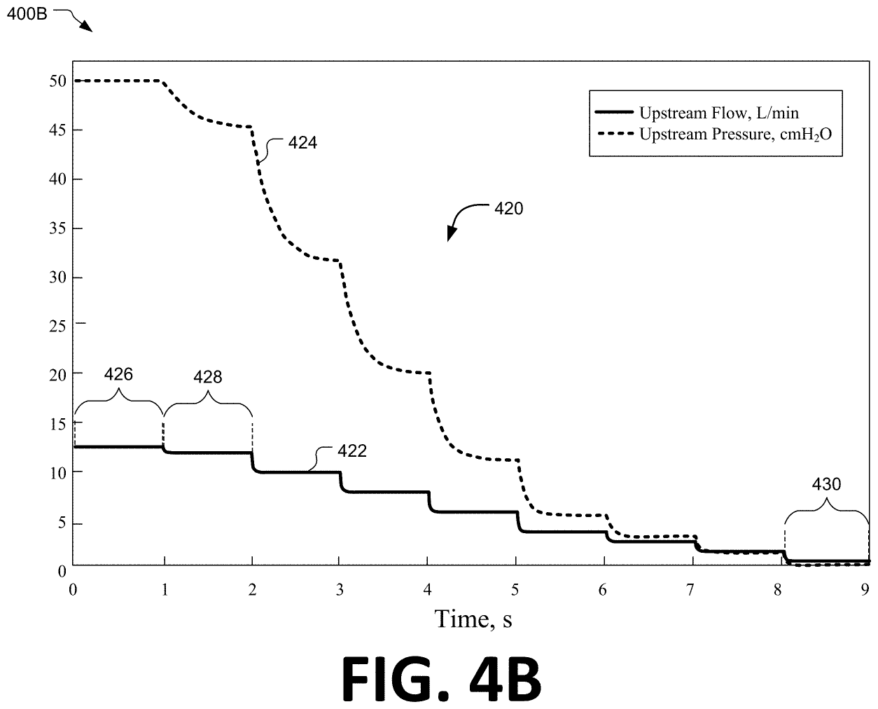

To implement a synchronized high-flow mode, a resistance of the breathing circuit may be estimated or otherwise determined. For instance, a resistance model for a breathing circuit may be determined by a calibration procedure. FIG. 4A shows an example method 400A for determining the circuit resistance, R_{HF}, of a single-limb breathing circuit based on a measured upstream flow and a measured upstream pressure, as may be used according to the disclosed synchronized high-flow system. The breathing circuit may include inhalation limb ventilation tubing and a non-invasive patient interface, such as a nasal cannula. The example method 400A includes operations that may be implemented or performed by the systems and devices disclosed herein. For example, the ventilators 100, 202, 304 depicted in FIGS. 1-3 may perform the operations described in the methods described herein. In addition, instructions for performing the operations of the methods disclosed herein may be stored in a memory of the ventilator (e.g., system memory 208 described in FIG. 2).

Method 400A describes one example calibration process for determining the circuit resistance of a breathing circuit. The operations of method 400A may be carried out while the patient is disconnected from the breathing circuit. One or more operations of method 400A may be implemented on a ventilator display, such as display 122, which allows performance of this calibration with a selection of a GUI interface button by a user.

The method 400A begins at operation 402 where a flow at a peak flow value, Q_{cal,max}, is commanded for a first time interval through a breathing circuit. A ventilator may command the peak flow. When a ventilator “commands” a flow value, a flow valve (e.g., flow valve 306) setting is adjusted such that the measured flow at a flow meter (e.g., flow meter 308) is the flow value. The flow or pressure “delivered” to the patient is referred to herein as the flow or pressure at the patient interface. The peak flow may be predetermined or may be selected by a clinician. In an example, the peak flow may be the same for any breathing circuit or, alternatively, may depend on the aspects of the breathing circuit or the intended patient, such as size of tubing, length of tubing, type of patient interface, predicted body weight (PBW) of patient, age of patient, etc. The peak flow value may be based on the flow command at which a predetermined pressure is measured. For example, the peak flow value may be associated with the flow command at which an inhalation pressure of 50 cmH₂O is measured. The peak flow value may be the highest flow value commanded during the calibration procedure.

At operation 404, the peak flow value and a peak pressure value are measured at the end of the first time interval. The ventilator may measure the peak flow value and the corresponding peak pressure value. For example, the ventilator may measure the peak flow value and the peak pressure value at one or more sensors, such as a flow meter (e.g., flow meter 308 to measure the inhalation flow, Qᵢ) and/or a pressure gauge (e.g., pressure gauge 310 to measure the inhalation pressure, Pᵢ). The measurement sensors used during operation 404 may be the same sensors used by the ventilator during measurement of upstream flow and upstream pressure during ventilation in a synchronized high-flow mode, as described herein. The first time interval may be based on how long the pressure takes to adjust to a change in commanded flow. This adjustment time may be based on the difference between the peak flow value and the prior flow command. For example, a greater change in flow command may increase the amount of time required (i.e., a longer first time interval) for the pressure to reach a sustained value (otherwise referred to as steady state). The peak flow value and the peak pressure value may be measured concurrently. Additionally, the flow and pressure may be measured several times over the duration of the first time interval.

At operation 406, the measured peak flow value and peak pressure value are added to a set of flow and pressure values (i.e., set of inhalation flows Qᵢ and set of inhalation pressures Pᵢ measured during calibration at different commanded flows). The set of flow and pressure values may be stored by the ventilator. The set of flow and pressure values may be stored temporarily or permanently. For example, the set of flow and pressure values may be deleted after calibration of the circuit resistance. The set of flow and pressure values may be stored in a matrix. In an instance where a plurality of flow values and pressure values are measured over the first time interval, one representative peak flow value and one representative peak pressure value may be added to the set of flow and pressure values, such as the last recorded peak flow value and peak pressure value in the first time interval. Alternatively, the representative values for the first time interval may be the peak flow value and peak pressure value measured two or more times without changing (e.g., a point in time during the first time interval where the measure pressure for the commanded flow reaches steady state).

At operation 408, subsequent to the first time interval, the flow is commanded at a decreased flow value for a second time interval. The ventilator may decrease the commanded flow to the decreased flow value by a step-down decrement. In an example, the step-down decrement is 1 L/min or 2 L/min. The second time interval may be the same or different than the first time interval. The second time interval may be based on the length of time until the pressure reaches steady state for the command flow. The second time interval may be based on the size of the step-down decrement.

At operation 410, at the end of the second time interval, the decreased flow value and a decreased pressure value are measured. Operation 410 may be similar to operation 404, except for measuring the decreased flow value and the decreased pressure value instead of the peak flow value and the peak pressure value. At operation 412, the measured decreased flow value and decreased pressure value are added to the set of flow and pressure values. Operation 412 may be similar to that of operation 406 except for adding the decreased flow value and the decreased pressure value to the set, instead of the peak flow value and the peak pressure value.

Operations 408-412 may repeat as required or desired for additional intervals. The step-down decrement may be the same or different for each time interval. Each time interval duration may be the same or different, and the durations of the time intervals may be based on the length of time required for the pressure to reach steady state for the decreased flow command. Operations 408-412 may repeat until the next decreased flow value is non-positive. For example, the peak flow value may be commanded to 12 L/min. The first step-down decrement may be 1 L/min, thus resulting in 11 L/min for the first decreased flow value. The second step-down decrement may be 2 L/min, thus resulting in 9 L/min for the second decreased flow value. In an instance where the remaining step-down decrements are 2 L/min, the resulting third through sixth decreased flow values are 7 L/min, 5 L/min, 3 L/min, and 1 L/min. For each commanded decreased flow, the flow value and pressure value for each time interval are added to the set of flow and pressure values.

In another example, the flow commanded may follow a predetermined sequence. For example, the flow commanded steps down in the following sequence, beginning with the value below the prior commanded flow (in L/min): 80, 60, 40, 30, 25, 20, 15, 12, 10, 8, 6, 4, 3, 2, 1. For instance, if the peak calibration flow is 12.5 L/min, then the subsequent flow steps, following the sequence, is 12 L/min, 10 L/min, 8 L/min, 6 L/min, 4 L/min, 3 L/min, 2 L/min, and 1 L/min. A predetermined sequence of flow commands limits the maximum amount of time required for a calibration procedure. For the example sequence above, which includes 15 sequence values, if each flow is commanded for a maximum of 1 second, then the calibration sequence spans a maximum total of 16 seconds (in a case where the first, peak flow value is not one of the numbers in the sequence and is greater than 80 L/min). In a case where 80 L/min is the maximum possible delivered flow, then the calibration sequence spans a maximum of 15 seconds if the peak flow value is greater than 60 L/min.

After operations 402-412, operation 414 determines a resistance model (e.g., R_{HF} 314) for the breathing circuit (e.g., breathing circuit 312), based on the set of flow and pressure values. The resistance model may be determined by the ventilator. With the set of flow and pressure values, the ventilator may identify relationships between the circuit resistance (as modeled by the resistance model) and the corresponding measured inhalation flow, Qᵢ, and inhalation pressure, Pᵢ. The resistance model may be non-linear. The resistance model for the breathing circuit, R_{HF}, may include coefficients, as described below:

$${R}_{HF} = \frac{{P}_{i}}{{Q}_{i}} = {β}_{0} + {β}_{1} \cdot {Q}_{i} \qquad ( Eqn . 1 )$$

where β₀ and β₁ are Rohrer's constants (also referred to herein as resistance constants). Rohrer's constants come from Rohrer's equation, an empiric model for airway resistance. It is expressed as:

R=β₀+β₁×Q (Eqn. 2)

where R is the resistance, Q is the volumetric flow rate, β₀ is the coefficient of laminar flow, and β₁ is a coefficient of turbulent flow.

Other models may include, for example, higher order or higher fidelity models based on electrical resistance theories, or lookup tables based on interpolation of values, or various types of mathematical models based on the physical law (another example is the Hagen-Poiseuille equation describing the pressure drop in a fluid flowing through a long cylindrical pipe).

At operation 416, an interface pressure model is generated based on the resistance model. The pressure drop across the breathing circuit may be related to the amount of resistance of the breathing circuit. The pressure gradient across the breathing circuit is represented as follows:

ΔP=R_{HFNC}·Qᵢ (Eqn. 3)

where ΔP is the pressure gradient across the breathing circuit. This equation for the pressure gradient (Eqn. 3) across the calibrated breathing circuit may be used to determine the interface pressure at the patient's nares, Pₙₐᵣₑₛ, which is modeled as follows:

Pₙₐᵣₑₛ=Pᵢ−ΔP=Pᵢ−R_{HFNC}·Qᵢ (Eqn. 4)

By substituting the resistance model, R_{HF}, described above in Eqn. 1, the interface pressure model may be written as follows:

Pₙₐᵣₑₛ=Pᵢ−(β₀+β₁·Qᵢ)·Qᵢ (Eqn. 5)

Using Eqn. 5, the interface pressure, Pₙₐᵣₑₛ, may be modeled during ventilation. For example, a calibration procedure may be performed to determine the Rohrer's constants, β₀ and β₁, for the breathing circuit. After the Rohrer's constants are known, the interface pressure may be estimated based on a measured inhalation flow, Qᵢ, and measured inhalation pressure, Pᵢ (e.g., as measured at a flow meter 308 and a pressure gauge 310).

In an example, a calibration procedure is executed for each interface that is used with the patient. That is, if a first interface (such as a nasal mask) is switched with a second different interface (such as a differently sized mask, or a nasal cannula), then the calibration routine is re-executed to obtain new Rohrer's constants for the new interface. Changing the interface without re-calibration may lead to inaccurate delivered interface pressure estimations unless the calibration process has been previously performed for the new interface. For instance, the Rohrer's constants may be predetermined and/or selectable based on characteristics of the breathing circuit, such as tubing length, tubing size, patient interface type, patient interface size, etc. Predetermined and/or selectable Rohrer's constants may be stored in the memory of the ventilator (e.g., memory 208 of ventilator 202) and/or retrievable from a remote resource via the internet or other network connection.

FIG. 4B shows an example of a graphical representation 400B of parameters measured or collected during the calibration procedure 420 of FIG. 4A. The graphical representation 400B shows the upstream flow 422 and the upstream pressure 424, during the calibration procedure 420.

As shown, the example calibration procedure includes a first time interval 426, a second time interval 428, and multiple other time intervals through the nth time interval 430. The first time interval 426 may have aspects of the first time interval described for operation 402. For example, the first time interval 426 may be associated with a flow commanded at a peak flow value, Q_{cal,max}. After the associated peak pressure value reaches steady state for the first time interval 426, the peak flow value and the peak pressure value may be measured and added to a set of flow and pressure values, as described herein.

The second time interval 428 and the nth time interval 430 may have aspects of the second time interval described for operation 408, as may be repeated. For example, the second time interval 426 and/or the nth time interval 430 may be associated with a flow commanded at a decreased flow value. After the associated decreased pressure value reaches steady state for the second time interval 428 or nth time interval 430, the decreased flow value and the associate decreased pressure value may be measured and added to the set of flow and pressure values, as described herein.

In the example shown in FIG. 4B, each time interval (including first time interval 426, second time interval 428, and nth time interval 430) spans 1 second. For this example breathing circuit calibration, the peak flow value is 12.5 L/min to achieve an upstream pressure 424 of 50 cmH₂O. The step-down decrement for the upstream flow 422 of the second time interval 428 is determined based on a predetermined sequence (e.g., in L/min, 80, 60, 40, 30, 25, 20, 15, 12, 10, 8, 6, 4, 3, 2, 1). The next value in the sequence, 12 L/min, is the upstream flow 422 for the second time interval 428. The upstream pressure 424 is measured at the end of the second time interval 428, after reaching steady state for the upstream flow 422 of 12 L/min. The upstream flow 422 is stepped down, based on the predetermined sequence, until the last, nth time interval 430 with an upstream flow 422 of 1 L/min, measuring the upstream pressure 424 at the end of the nth time interval 430 after the upstream pressure 424 has reached steady state.

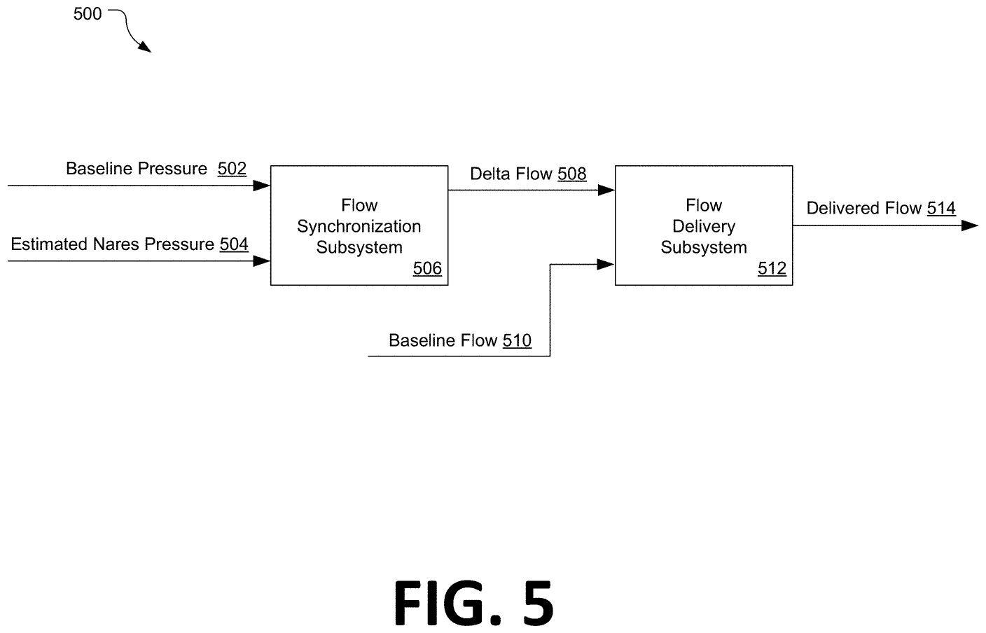

FIG. 5 is a block diagram illustrating an example system 500 for implementing a synchronized HF. The system 500 includes a flow synchronization subsystem 506 and a flow delivery subsystem 512.

The flow synchronization subsystem 506 receives as inputs a baseline pressure 502 and an estimated nares pressure 504. The baseline pressure 502 may be any non-negative (i.e., greater than or equal to zero) pressure to be delivered to the patient through the patient interface in the breathing circuit. The baseline pressure 502 may be selected manually by a clinician, or may be automatically determined based on one or more patient parameters, such as PBW, age, lung condition, etc. For example, the baseline pressure may be equal to or based on a PEEP value. In another example, the baseline pressure 502 may be zero.

As described herein, the estimated nares pressure 504 (otherwise referred to as estimated interface pressure 504, or interface pressure 504) may be determined based on a measured inhalation flow (e.g., upstream flow measured at flow meter 308 or upstream flow 422), a measured inhalation pressure (e.g., upstream pressure measured at pressure gauge 310 or upstream pressure 424), and a circuit resistance model (e.g., circuit resistance, R_{HF}, 314). In an example, the estimated nares pressure 504 may not be calculated directly and, instead, the inhalation flow, inhalation pressure, and resistance model may be provided to the flow synchronization subsystem 506.

The flow synchronization subsystem 506 may determine a delta flow 508 based on a comparison of the baseline pressure 502 with the estimated nares pressure 504. The delta flow 508 is a function of the difference between the baseline pressure 502 and the estimated nares pressure 504. If the estimated nares pressure 504 is less than the baseline pressure 502, then the flow synchronization system 506 may determine a delta flow 508 to compensate for (or reduce) the difference between the baseline pressure 502 and the estimated nares pressure 504. Based on the relationships described above, an increase in the commanded flow increases the estimated nares pressure. Thus, to increase the estimated nares pressure 504 up to a baseline pressure 502, the inhalation flow may be increased. The amount of increase in flow required to increase the estimated nares pressure 504 to substantially the baseline pressure 502, or the delta flow 508, is calculated by the flow synchronization system 506.

The delta flow 508, as determined by the flow synchronization subsystem 506, is received as an input at the flow delivery subsystem 512. Additionally, the flow delivery subsystem 512 receives a baseline flow 510. The flow delivery subsystem 512 adds the delta flow 508 to the baseline flow 510 to augment the inhalation flow delivered to the patient. If the estimated nares pressure 504 is equal to or greater than the baseline pressure 502, then there is no need to augment the inhalation flow. In this instance, where the estimated nares pressure 504 is at least the baseline pressure 502, then the flow synchronization system 506 may set the delta flow 508 equal to zero. When the delta flow equals zero, the inhalation flow is maintained at the same, constant value as previously delivered. Thus, the flow synchronization subsystem 506 calculates a delta flow 508 sufficient to adjust the estimated nares pressure 504 to be substantially (or within an acceptable error or tolerance) at least the baseline pressure 502. In other words, the flow through the patient interface is augmented by the flow delivery subsystem 512 when the patient inhales more breathing gases than being provided at the patient interface at the baseline pressure 502 such that the flow through the patient interface is “synchronized” with the patient's desired inhalation when the inhalation exceeds the baseline flow 510 provided at the baseline pressure 502.

The baseline flow 510 is continuously delivered during active ventilation in a synchronized high-flow mode. The baseline flow 510 may be selected by a clinician or the ventilator. Additionally, the baseline flow 510 may be based on a desired flow or desired pressure at the patient interface. When the flow synchronization system 506 determines that the delta flow 508 is greater than zero, the flow delivery system 512 augments the baseline flow 510 with the delta flow 508. Thus, the flow delivery system 512 delivers an augmented or synchronized delivered flow 514 equal to the delta flow 508 plus the baseline flow 510. The delivered flow 514 is therefore at least the baseline flow 510 and is greater than the baseline flow 510 when the delta flow 508 is greater than zero.

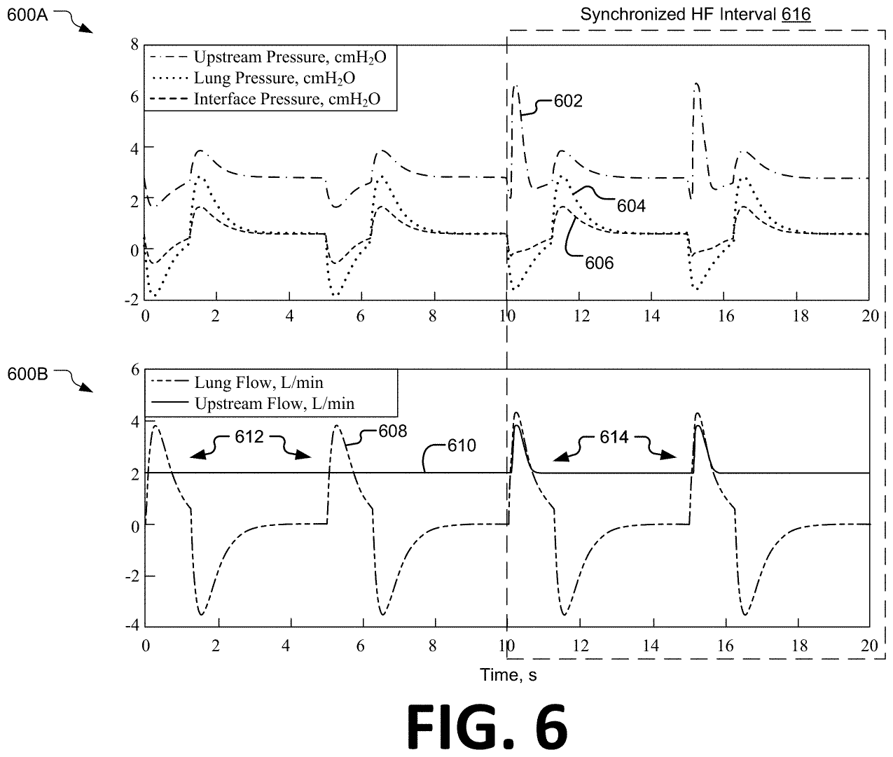

FIG. 6 shows a graphical example 600A of pressure parameters measured or collected during a simulated ventilation with a high-flow system with an HFNC patient interface, and a corresponding graphical example 600B of flow parameters measured or collected during ventilation with the high-flow system. The pressure parameters shown in graphical example 600A include an upstream pressure 602 (i.e., a pressure measured upstream of the breathing circuit), a lung pressure 604 (i.e., a pressure in the lungs of the patient), and an interface pressure 606 (i.e., the pressure at the patient interface, such as the pressure at the anterior tips of a nasal cannula or the nares pressure when using an HFNC). The flow parameters shown in graphical example 600B include a lung flow 608 (i.e., a flow in out of the lungs of the patient) and an upstream flow 610 (i.e., a flow measured upstream of the breathing circuit). As further shown in the graphical example 600B, there two non-synchronized flow breaths 612, and two synchronized flow breaths 614 during the synchronized high-flow interval 616, utilizing the synchronized high-flow mode described herein. As referenced herein, a flow deficiency occurs when the lung flow 608 of the patient exceeds the baseline flow (here, 2 L/min) of the upstream flow 610. During a flow deficiency, if the delivered flow is not compensated (such as during a synchronized high-flow mode), then the patient draws additional air from the room, in addition to the breathing gases received from at the patient interface, thereby diluting the breathing gases delivered at the patient interface. For example, if ventilating a patient with a nasal cannula, when the patient inhales at a higher rate than the flow provided at the patient interface, room air is inhaled by the patient to compensate for the flow difference. Inhalation of room air may reduce the concentration of oxygen delivered to the patient if the breathing gases have a higher oxygen concentration than the room air. Additionally, inhalation of room air may cause inaccurate measurements for partial pressure of CO₂. Additionally, if the patient is breathing in room air, the patient may not receive some of the benefits of high-flow systems. For example, the patient may experience improper washout of the nasopharyngeal dead space or suboptimal gas conditioning.

Without using the synchronized high-flow mode aspects described herein (e.g., as shown prior to the synchronized high-flow interval 616), ventilation parameters may be similar to the parameters shown in the graphical examples 600A, 600B during the non-synchronized flow breaths 612. Specifically, without synchronizing flow through the high-flow system, the upstream flow, or the upstream flow 610 remains constant and unchanged. In the example shown, the upstream flow 610 remains at a constant 2 L/min for the duration of ventilation, including for the duration of the non-synchronized flow breaths 612. The pressure measurements of graphical example 600A reflect the changes in various pressures resulting from a constant upstream flow 610 prior to the synchronized high-flow interval 616 (e.g., between 0 seconds and 10 seconds). For example, as the beginning of the inhalation phase, the upstream pressure 602, the lung pressure 604, and the interface pressure 606 sharply decrease, followed by a slow increase in pressures through the remainder of the inhalation phase. At the beginning of the exhalation phase, the pressures experience a sharp increase, followed by a slow decrease that levels off toward the end of the exhalation phase. The pressures then experience a sharp decrease as the patient transitions back into the start of the inhalation phase.

In some instances (for some patients), such as the example shown in FIG. 6, the decrease in pressures at the start of the inhalation phase may cause the interface pressure 606 at the patient interface to drop below a baseline pressure (e.g., zero cm H₂O). As described above, a drop in the interface pressure or the interface pressure 606 below the baseline pressure may not be optimal for the patient (e.g., the flow demanded by the patient may be higher than the flow desired to be provided to the patient).

In contrast, during the synchronized high-flow interval 616, the upstream flow 610 is augmented to prevent the interface pressure 606 from dropping too far below a baseline pressure (in this example, the baseline pressure is zero cmH₂O). During the synchronized flow breaths 614, the upstream flow 610 is increased above a baseline flow (in this example, 2 L/min) to prevent a drop in the interface pressure 606 below the baseline pressure (here, zero cmH₂O), thus providing an upstream flow 610 similar to the lung flow 608 during the synchronized flow breaths 614. The increase in the upstream flow 610 during the synchronized high-flow interval 616 may be based on a resistance model and/or interface pressure model determined for the breathing circuit during calibration (such as the calibration procedure described in FIGS. 4A-B). For example, when the measured upstream pressure 602 sharply decreases at the beginning of the inhalation phase, the estimated interface pressure 606 also sharply decreases. To prevent the estimated interface pressure 606 (Pₙₐᵣₑₛ in the above equations) from dropping too far below a baseline pressure (in this example, non-negative), the upstream flow 610 (Qᵢ in the above equations) may be increased, increasing the upstream pressure 602 (Pᵢ in the above equations) and also increasing the interface pressure 606. Thus, the upstream flow 610 may be “synchronized” with the patient's lung flow 608 when the lung flow 608 is greater than the baseline flow (here, 2 L/min, as shown at the synchronized flow breaths 614).

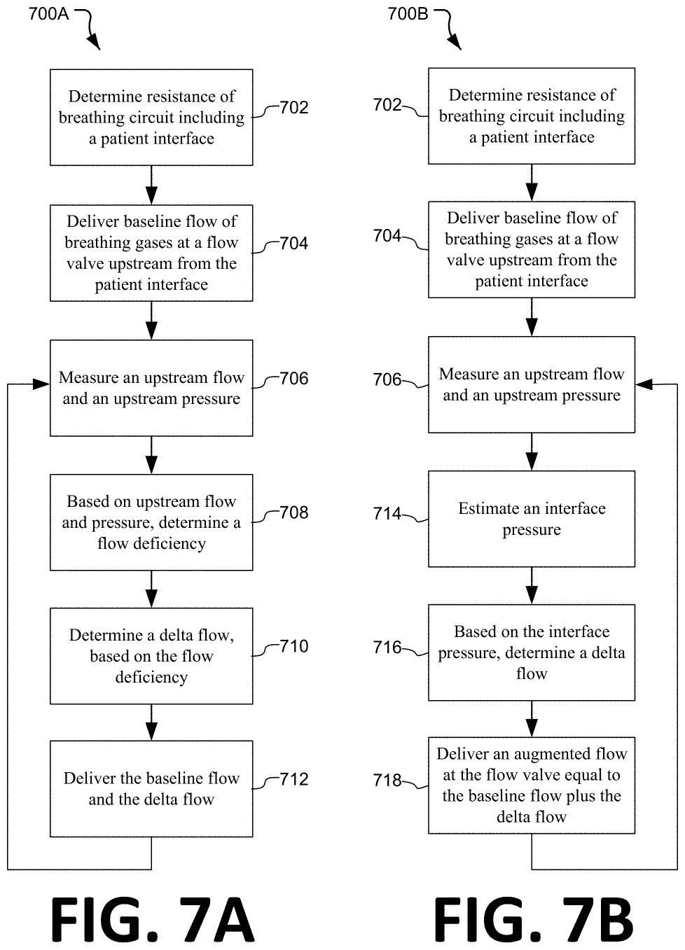

FIGS. 7A-B, 8, 9, and 10 show example methods according to the disclosed synchronized high-flow mode. The example methods include operations that may be implemented or performed by the systems and devices disclosed herein. For example, the ventilators 100, 202, 304 depicted in FIGS. 1-3 may perform the operations described in the methods. In addition, instructions for performing the operations of the methods disclosed herein may be stored in a memory of the ventilator (e.g., system memory 208 described in FIG. 2).

FIGS. 7A-B show example methods 700A, 700B of synchronized high-flow mode. The methods 700A, 700B shown in FIGS. 7A-B share operations 702-706 but diverge after operation 706. The methods 700A, 700B begin at operation 702, where a resistance of the breathing circuit, including a patient interface, is determined. A resistance for the breathing circuit may be based on a model. For example, the resistance may be the same as the resistance models described herein (e.g., resistance model, R_{HF}, 314 in FIG. 3 or the resistance model described at operation 414 of FIG. 4A). The breathing circuit may have characteristics described herein (e.g., breathing circuit 312), including a tubing system (e.g., ventilation tubing system 130) and a patient interface (e.g., patient interface 180). The patient interface may be any non-invasive interface that delivers breathing gases to the nares of the patient, such as a nasal cannula or nasal mask. The breathing circuit may be single limb. In addition to the tubing system and the patient interface, the breathing circuit may also include a humidifier.

At operation 704, a baseline flow of breathing gases is delivered at a flow valve upstream from the patient interface. The baseline flow may be selected by a clinician or by the ventilator. The baseline flow may be based on a patient parameter, such as PEEP, lung condition, PBW, respiratory rate, desired tidal volume, age, etc. Additionally or alternatively, the baseline flow may be associated with a baseline pressure delivered at the patient interface when the patient is disconnected from the interface. For example, the resistance model at operation 702 may be used to determine a baseline flow to cause a specified or predetermined baseline pressure to be delivered at the patient interface. The breathing gases may be provided to the ventilator from a pressurized source, such as a wall or compressed gas tank. The flow valve controls the flow of breathing gases from the pressurized source into the breathing circuit for delivery to the patient. The flow valve may be similar to the flow valves described herein (e.g., valves described in FIG. 1, and flow valve 306 described in FIG. 3). The flow valve may be positioned upstream of the breathing circuit, which includes the patient interface. In an example, the flow valve may be part of an inhalation module of the ventilator.

At operation 706, an upstream flow and an upstream pressure are measured. The upstream flow and the upstream pressure may be measured near, but downstream of, the flow valve. Additionally, the upstream flow and upstream pressure are measured upstream of the patient interface and/or substantially upstream of the breathing circuit (including a tubing system and the patient interface). The upstream flow may be measured using a flow meter (e.g., flow meter 308). The upstream pressure may be measured using a pressure gauge (e.g., pressure gauge 310). The measurements may be taken a specified time interval, such as every 5 ms.

Method 700A continues to operations 708-712, while method 700B continues to operations 714-718. At operation 708, based on the upstream flow and the upstream pressure, a flow deficiency is determined. As further described herein, a flow deficiency occurs when the lung flow of the patient (or flow rate of gas being inhaled at the patient's nares) exceeds the baseline flow. The baseline flow may account for a desired positive end expiratory pressure. Thus, the flow deficiency changes over the length of a patient's breath. As further described at least with respect to FIGS. 5-6, the flow deficiency may be associated with a pressure estimated at the patient interface dropping below a baseline pressure, as flow and pressure are related using the resistance model. The flow deficiency may be binary (e.g., there is or is not a flow deficiency). In an example, the flow deficiency may be considered negligible if the flow deficiency is less than a threshold deficiency. The flow deficiency may be determined using the interface pressure model for the breathing circuit (such as Eqn. 5, above).

At operation 710, a delta flow is determined based on the flow deficiency. If there is no flow deficiency (i.e., the lung flow rate is less than or equal to the baseline flow), then the delta flow is zero. In the presence of a flow deficiency, the delta flow may be calculated by the ventilator. The delta flow may be a value less than or equal to the flow deficiency. To prevent overcompensating, the delta flow may be limited to a percentage of the flow deficiency. Additionally, the delta flow may be set to zero when the flow deficiency is below a threshold. Alternatively, the delta flow may be based on a predetermined step-up increment based on the flow deficiency. As the flow deficiency varies over the course of the patient's breath, the delta flow varies. For example, the delta flow may increase or decrease over the course of a breath but have a value that is greater than or equal to zero.

At operation 712, the baseline flow and the delta flow are delivered. Since the delta flow is greater than or equal to zero, the minimum flow delivered is the baseline flow. Thus, the delta flow may augment the total delivered flow above that of the baseline flow. Delivery of the baseline flow and the delta flow may be accomplished by changing a flow command. The change in flow command may change a setting of the flow valve to increase or decrease the amount of flow delivered to the patient through the breathing circuit.

Operations 706-712 may repeat as required or desired. For example, operations 706-712 may repeat for each measured upstream flow and upstream pressure. As described herein, the upstream flow and the upstream pressure may be measured at set interval. Thus, operations 706-712 may repeat for each new measurement of upstream flow and upstream pressure for each measurement interval.

Alternatively, following method 700B, at operation 714, an interface pressure is estimated. An interface pressure may be estimated based on the resistance determined at operation 702, in addition to the measured upstream flow and upstream pressure, as further described herein. Thus, the interface pressure changes with the upstream flow and the upstream pressure according to the resistance model for the breathing circuit.

At operation 716, based on the interface pressure, a delta flow is determined. To determine a delta flow, the interface pressure may be compared to a desired baseline pressure at the patient interface. When the interface pressure is less than the baseline pressure, then the patient is demanding more breathing gases than are intended to be provided at the patient interface. A delta flow is then determined, based on the interface pressure and the baseline pressure such that, if the baseline flow were increased by the delta flow, the pressure difference between the interface pressure and the baseline pressure is reduced. The delta flow may be calculated based on predetermined step increments, as a portion of the flow required to increase the interface pressure to the baseline pressure, as a percentage of the pressure difference between the interface pressure and the baseline pressure, or other method of selecting a delta flow. If the interface pressure is greater than or equal to the baseline flow, the delta flow may be set to zero.

At operation 718, an augmented flow is delivered, at the flow valve, equal to the baseline flow plus the delta flow. The delta flow is used to increase the delivered flow over the baseline flow, thus, the delta flow is generally greater than or equal to zero (or non-negative).

Operations 706 and 714-718 may repeat as required or desired. For example, operations 706 and 714-712 may repeat for each measured upstream flow and upstream pressure. As described herein, the upstream flow and the upstream pressure may be measured at set interval. Thus, operations 706 and 714-718 may repeat for each new measurement of upstream flow and upstream pressure for each measurement interval. For example, a first upstream flow and first upstream pressure are measured. Based on the first upstream flow and first upstream pressure, a first interface pressure is estimated. Based on the first interface pressure, a first delta flow is determined. A first augmented flow is delivered equal to the baseline flow plus the first delta flow. Subsequent to delivering the first augmented flow, a second upstream flow and a second upstream pressure are measured. Based on the second upstream flow and second upstream pressure, a second interface pressure is estimated. Based on the second interface pressure, a second delta flow is determined. A second augmented flow is delivered equal to the baseline flow plus the second delta flow.

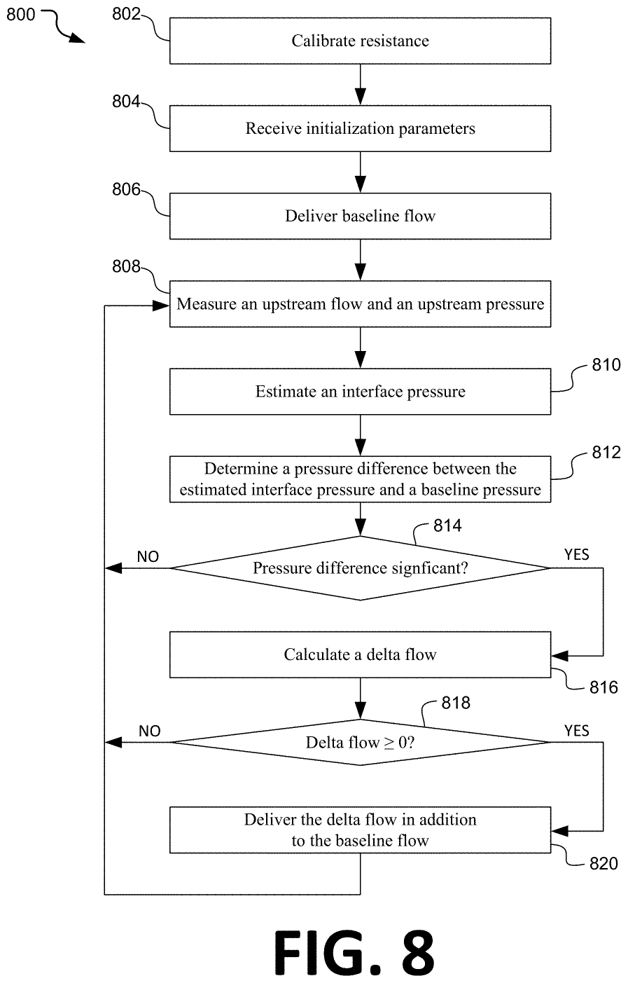

FIGS. 8, 9, and 10 are additional example methods 800, 900, 1000 of implementing a synchronized high-flow mode. For example, FIG. 8 shows an example method 800 for a synchronized high-flow mode. Method 800 begins at operation 802 where a resistance is calibrated. Resistance calibration for the breathing circuit may be the same as that described for the calibration process in FIGS. 4A-B.

At operation 804, initialization parameters are received. Initialization parameters may include a baseline flow, a baseline pressure, a deficiency threshold, and/or gain value. The baseline flow and the baseline pressure may be the same as further described herein. The deficiency threshold is the threshold at which, or above which, the delivered flow should be increased (such as by a delta flow). Gain value may dictate how much the delivered flow is increased for a period of time. The gain value may be implemented to prevent overcompensated adjustment of the delivered flow and/or to prevent lung injury (e.g., if the flow and/or pressure at the patient interface is too high, or if the flow and/or pressure at the patient interface change too quickly).

At operation 806, a baseline flow is delivered. The ventilator may deliver the baseline flow as determined or received at operation 804. At operation 808, an upstream flow and an upstream pressure are measured. The upstream flow and/or the upstream pressure may be measured in the same manners as those described herein (e.g., operation 706 in FIGS. 7A-B). At operation 810, an interface pressure is estimated. The interface pressure may be estimated in the same or similar way as that described for operation 714 in FIG. 7B. For example, the interface pressure may be determined based on a resistance of the breathing circuit and the measured upstream flow and upstream pressure.

At operation 812, a pressure difference between the estimated interface pressure and a baseline pressure is determined. The pressure difference may be determined by the ventilator by subtracting the baseline pressure from the interface pressure estimated in operation 810.

At determination 814, the pressure difference is evaluated for significance. In an example, the pressure difference may be determined to be significant when the absolute value of the pressure difference meets or exceeds a threshold. Alternatively, the pressure difference may be determined to be significant when the pressure difference falls outside of a threshold range. Accordingly, determination 814 may include comparing the pressure difference determined in 812 to a threshold. The factors for determining if the pressure difference is significant may be established along with the parameters received at operation 804. In examples, the determination 814 is binary (e.g., whether the pressure difference is significant) or may be based on a threshold (e.g., whether the pressure different is at least or at most a specified amount). If it is determined that the pressure difference is not significant, flow branches “NO” back to operation 808. Thus, operations 808-814 may repeat as required or desired until the pressure difference is significant.

If the pressure difference is determined to be significant in operation 814, the method 800 branches “YES” to operation 816 where a delta flow is calculated. The delta flow may be determined or calculated as further described herein (e.g., as described in FIG. 5 or described at operation 716 in FIG. 7B). As descried herein, a flow deficiency occurs when the pressure difference (i.e., the interface pressure minus the baseline pressure) is negative. In other words, when the estimated interface pressure is less than the baseline pressure, a flow deficiency may be present. Thus, the delta flow is a positive value to increase the flow when a flow deficiency is present. If the interface pressure is greater than the baseline pressure, the calculated delta flow value may be negative. In some examples, the delta flow value may be proportional to the pressure difference determined in operation 814. For instance, a greater pressure difference results in a greater delta flow value. Additionally or alternatively, the delta flow may be calculated as a function of the pressure difference and the measured inhalation flow or upstream flow, Qᵢ. For example, delta flow may be calculated as the pressure difference divided the resistance (which is a function of Qᵢ).

At determination 818, it is determined if the delta flow is greater than or equal to zero (otherwise referred to herein as non-negative), which occurs when the interface pressure less than or equal to the baseline pressure. In examples, the determination 818 is binary (e.g., whether the delta flow is negative or non-negative) or may be based on a threshold (e.g., whether the delta flow is at least a specified value). If it is determined that the delta flow is negative (or not non-negative), flow branches to “NO” back to operation 808. Thus, operations 808-818 may repeat as required or desired until the delta flow is non-negative.

If, however, it is determined that the delta flow is non-negative, flow branches “YES” to operation 820, where the delta flow is delivered in addition to the baseline flow. The combined delta flow and the baseline flow may be referred to herein as an augmented flow. Thus, when delta flow is non-negative the delivered flow is the baseline flow plus the delta flow, and when delta flow is negative the delivered flow is the baseline flow. The ventilator may automatically update ventilator settings to adjust or augment the delivered flow based on the delta flow, as described herein.

Operations 808-820 may repeat as required or desired. For example, operations 808-820 may repeat for each measured upstream flow and upstream pressure. As described herein, the upstream flow and the upstream pressure may be measured at set interval. Thus, operations 808-820 may repeat for each new measurement of upstream flow and upstream pressure for each measurement interval.

In an example, the baseline pressure is zero cmH₂O and the deficiency threshold is 0.1 cmH₂O. At a first measurement set, a first upstream flow and a first upstream pressure are used to estimate a first interface pressure of 0.7 cmH₂O. The first pressure difference (the interface pressure minus the baseline pressure) is 0.7 cmH₂O, the absolute value of which exceeds the deficiency threshold of 0.1 cmH₂O. Because the first pressure difference indicates that the interface pressure is greater than or equal to the baseline pressure, no flow deficiency exists, and no additional flow is provided. At a second measurement set, a second upstream flow and a second upstream pressure are associated with a second interface pressure of −0.2 cmH₂O. The second pressure difference is −0.2 cmH₂O, the absolute value of which exceeds the deficiency threshold. Because the second pressure difference indicates that the interface pressure is less than the baseline pressure, the second delta flow is greater than zero. At a third measurement set, a third upstream flow and a third upstream pressure are associated with a third interface pressure of −0.5 cmH₂O. The third pressure difference is −0.5 cmH₂O, the absolute value of which exceeds the deficiency threshold. Because the third pressure difference indicates that the interface pressure is less than the baseline pressure by more than the second pressure difference, the third delta flow is greater than zero and greater than the second delta flow. At a fourth measurement set, a fourth upstream flow and a fourth upstream pressure are associated with a fourth interface pressure of −0.3 cmH₂O. The fourth pressure difference is −0.3 cmH₂O, the absolute value of which exceeds the deficiency threshold. Because the fourth pressure difference indicates that the interface pressure is less than the baseline pressure by more than the second pressure difference and less than the third pressure difference, the fourth delta flow is greater than zero, greater than the second delta flow, and less than the third delta flow.

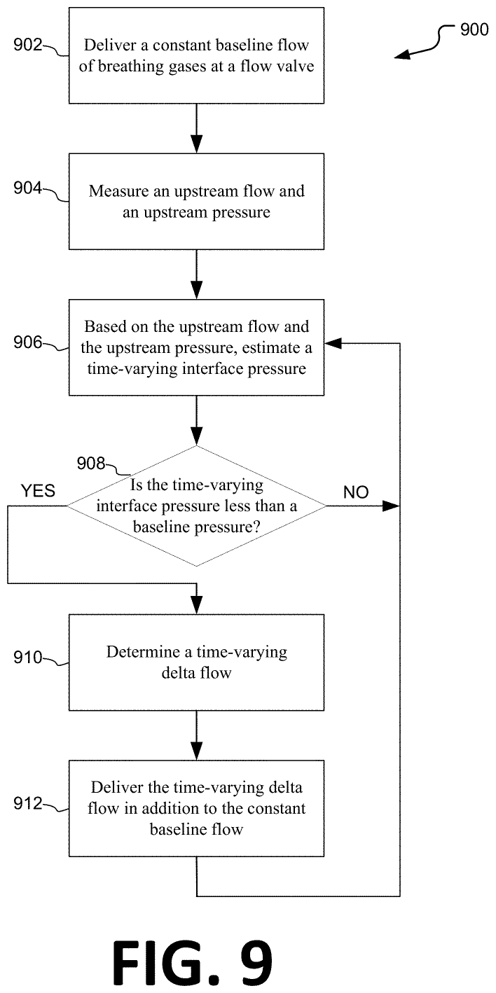

FIG. 9 shows another example method 900 for a synchronized high-flow mode. Method 900 begins at operation 902 where a constant baseline flow of breathing gases is delivered at a flow valve. The constant baseline flow may be correlated with a baseline pressure at a patient interface when a patient is disconnected from the breathing circuit. The constant baseline flow may be the minimum flow delivered by the ventilator throughout the course of active ventilation of the patient when the patient is connected to the patient interface. The constant baseline flow may be selected by a clinician or determined by the ventilator, as further described herein.

At operation 904, an upstream flow and an upstream pressure are measured. Operation 904 may the same as, or similar to, other measurement operations described herein (e.g., operation 706 or operation 808).

At operation 906, based on the upstream flow and the upstream pressure, a time-varying interface pressure is estimated. As the patient breathes, the patient demand for breathing gases varies. For example, as a patient inhales, the flow demanded at the patient interface increases, decreasing the interface pressure at the patient interface. Conversely, as the patient exhales, the flow demanded at the patient interface decreases, increasing the pressure at the patient interface. Thus, the interface pressure varies over time (a time-varying interface pressure), as the patient inhales and exhales, as may be unique from breath to breath. Measurements of the upstream flow and the upstream pressure at different points in time may be used to estimate the interface pressure at that point in time. Estimation of the interface pressure based on measured upstream flow and upstream pressure is further described herein.

At determination 908, the time-varying interface pressure (at a specific point in time) is evaluated against a baseline pressure. For example, if the time-varying interface pressure is less than the baseline pressure (described at operation 902). In examples, the determination 908 is binary (e.g., whether the time-varying interface pressure is less than the baseline pressure) or may be based on a threshold (e.g., whether the time-varying pressure is less than the baseline pressure by at least a specified amount). If it is determined that the time-varying interface pressure is not less than the baseline pressure (i.e., greater than or equal to the baseline pressure), flow branches “NO” back to operation 906. Operations 906-908 may repeat as required or desired until the time-varying interface pressure is less than the baseline pressure.

If, however, it is determined that the time-varying interface pressure is less than the baseline pressure, flow branches “YES” to operation 910, where a time-varying delta flow is determined. Determination of the time-varying delta flow for a specific point in time may be the same as, or similar to, determinations of delta flow as described herein (e.g., at operation 710 or at operation 716). At operation 912, the time-varying delta flow is delivered in addition to the constant baseline flow. Operation 912 may be the same as, or similar to, other delivery operations that include delta flow, as described herein (e.g., operations 712, 718, 820).

Operations 906-912 may repeat as required or desired. For example, operations 906-912 may repeat for each measured upstream flow and upstream pressure. As described herein, the upstream flow and the upstream pressure may be measured at set interval. Thus, operations 906-912 may repeat for each new measurement of upstream flow and upstream pressure for each measurement interval.

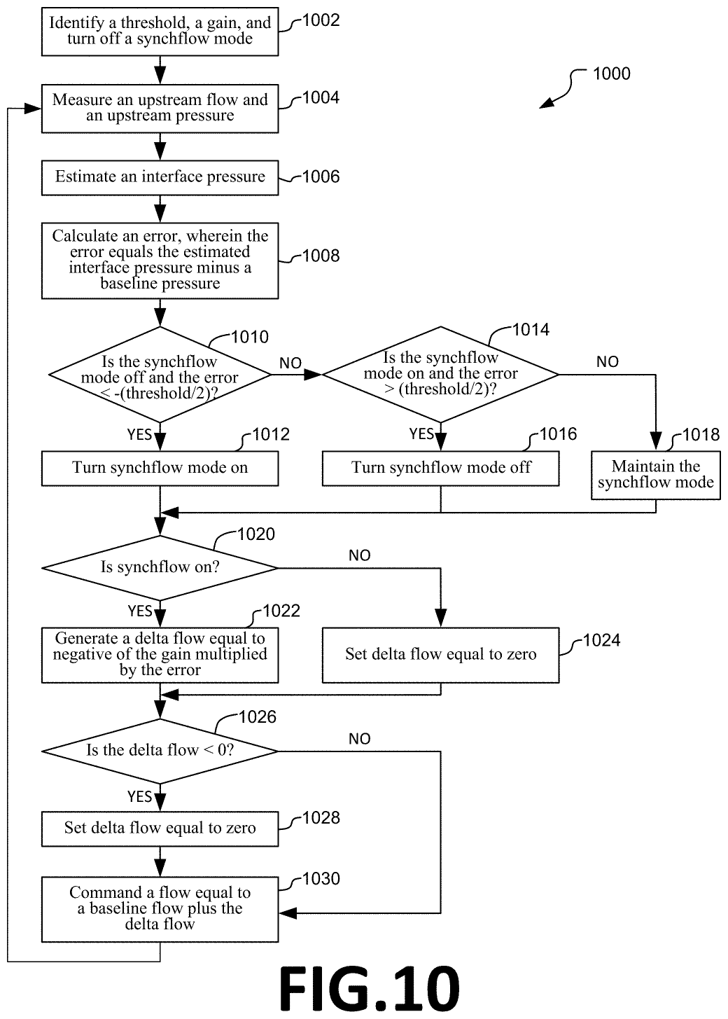

FIG. 10 shows another example method 1000 for a synchronized high-flow mode. Method 1000 begins at operation 1002 where a threshold and a gain are identified, and a synchflow mode is turned off. The threshold is a threshold pressure or a threshold pressure range inside which a pressure difference may be considered negligible or may be ignored to prevent unnecessary oscillations to compensate for small pressure differences. The threshold may be a hysteresis band. The gain is a proportionality constant or multiplier for increases to the delivered flow. Synchflow mode (or synchronized high-flow mode) is a mode that allows augmentation of the delivered flow to increase the delivered flow above a constant, baseline flow. When synchflow mode is off, the ventilator delivers the constant, baseline flow. When synchflow mode is on, the ventilator may deliver a delta flow in addition to the baseline flow, as further described in the following operations. Thus, at operation 1002, the ventilator is delivering a constant, baseline flow or has not yet initiated ventilation.

At operation 1004, an upstream flow and an upstream pressure are measured. Operation 1004 may be the same as, or similar to, other measurement operations described herein (e.g., operation 706, operation 808, or operation 904). At operation 1006, an interface pressure is estimated. Estimation of the interface pressure may be performed as described herein (e.g., operation 714, operation 810, or operation 906). At operation 1008, an error is calculated, wherein the error (otherwise referred to as the pressure difference) equals the estimated interface pressure minus a baseline pressure.

At determination 1010, the synchflow mode is determined to be off and the error is determined to be less than negative of half of the threshold (in an example where the threshold is a threshold pressure range). Alternatively, if the threshold is a threshold pressure value, the error evaluation may be if the error is less than negative of the threshold. If synchflow mode is off and the error is less than negative of half of the threshold (or, in some cases, the negative of the whole threshold), flow branches “YES” to operation 1012, where synchflow mode is turned on. When the error is significant (in this example, less than negative of half of the threshold), then the delivered flow should be augmented by the synchflow mode.

If, however, it is determined that either synchflow mode is on or the error is not greater than negative of half of the threshold, flow branches “NO” to determination 1014. At determination 1014, it is determined if the synchflow mode is on and if the error is greater than half the threshold. In other words, if synchflow mode is on and the baseline pressure is exceeding the interface pressure by at least half of the threshold, flow branches “YES” to operation 1016 where synchflow mode is turned off.

If, however, it is determined that either the synchflow mode is off or the error is less than half of the threshold, flow branches “NO” to operation 1018 where the current synchflow mode is maintained. For example, the synchflow mode was “on” prior to operation 1018, the synchflow mode would continue to be “on.” Alternatively, if the synchflow mode was “off” prior to operation 1018, the synchflow mode would continue to be “off.”

At determination 1020, it is determined if the synchflow mode is on. If it is determined that synchflow mode is off, flow branches “NO” to operation 1024 where the delta flow is set equal to zero. If, however, it is determined that synchflow mode is on, flow branches “YES” to operation 1022. At operation 1022, a delta flow that is equal to negative of the gain multiplied by the error is generated. As described above, when the error is negative, the estimated interface pressure is less than the baseline pressure and the flow should be increased (e.g., by an amount equal to the delta flow) to increase the interface pressure. As defined above, the gain is a proportionality constant or multiplier for increases to the delivered flow. If the gain is between zero and one, the delta flow is a portion of the entire error determined at operation 1008. By limiting the amplitude of the delta flow relative to the error, overcompensation of the delivered flow may be reduced or prevented.

At determination 1026, it is determined if the delta flow is less than zero (or negative). If it is determined that delta flow is less than zero (i.e., the interface pressure is greater than the baseline pressure, resulting in a pressure surplus at the patient interface and a negative delta flow), flow branches “YES” to operations 1028-1030. At operation 1028, the delta flow is set equal to zero. In this way, the delta flow does not augment the delivered flow to reduce the delivered flow below the baseline flow value (by adding a negative delta flow value at operation 1030). At operation 1030, a flow equal to a baseline flow plus the delta flow is commanded. If the delta flow was determined to be negative at determination 1026, the delta flow is reset to zero such that the commanded flow equal the baseline flow without augmentation.

If, however, it is determined at determination 1026 that the delta flow is greater than or equal to zero (i.e., the interface pressure is less than or equal to the baseline pressure, resulting in a pressure deficit at the patient interface and a non-negative delta flow), flow branches “NO” to operation 1030. Thus, the flow commanded at operation 1030 is at least the baseline flow during active ventilation, up to a commanded flow of the baseline flow plus a non-negative delta flow.

Operations 1004-1030 may repeat as required or desired. For example, operations 1004-1030 may repeat for each measured upstream flow and upstream pressure. As described herein, the upstream flow and the upstream pressure may be measured at set interval. Thus, operations 1004-1030 may repeat for each new measurement of upstream flow and upstream pressure for each measurement interval.

Although the present disclosure discusses the implementation of these techniques in the context of a ventilator capable of implementing a synchronized high-flow mode, the techniques introduced above may be implemented for a variety of medical devices or devices utilizing flow sensors. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Although this disclosure describes synchronizing flow through a nasal cannula in a synchronized high-flow mode, it should be appreciated that any type of patient interface capable of delivering high flow may be implemented. Additionally, although aspects of the disclosure describe estimating a patient interface pressure using a calibration procedure, methods and systems of the disclosure may instead be compatible with a direct pressure measurement at the patient interface, an estimation or measurement of flow at the patient interface, or other measurements, estimates, or characterizations at the patient interface to determine how the delivered flow should be augmented.

Those skilled in the art will recognize that the methods and systems of the present disclosure may be implemented in many manners and as such are not to be limited by the foregoing aspects and examples. In other words, functional elements being performed by a single component, or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for synchronizing flow delivered to a patient at a patient interface, the method comprising:
determining a resistance of a breathing circuit including a patient interface;
delivering a baseline flow of breathing gases at a flow valve upstream from the patient interface;
measuring an upstream flow and an upstream pressure, the upstream flow and the upstream pressure determined at a position between the flow valve and the patient interface;
estimating an interface pressure at the patient interface;
based on the interface pressure, determining a delta flow; and
delivering an augmented flow at the flow valve, wherein the augmented flow equals the baseline flow plus the delta flow.

2. The method of claim 1, wherein the baseline flow is delivered according to a synchronized high-flow mode.

3. The method of claim 1, wherein determining the resistance includes performing a calibration procedure by varying the upstream flow and the upstream pressure.

4. The method of claim 1, wherein estimating the interface pressure is based on the upstream flow, the upstream pressure, and the resistance.

5. The method of claim 1, wherein the patient interface is a nasal cannula and the interface pressure is estimated at nares of the nasal cannula.

6. The method of claim 1, wherein determining the delta flow is further based on a pressure difference between the interface pressure and a baseline pressure.

7. The method of claim 6, wherein the delta flow is equal to a non-negative value proportional to the pressure difference.

8. The method of claim 1, wherein the baseline pressure is zero.

9. The method of claim 1, wherein the delta flow is time-varying and non-negative.

10. A method for synchronizing flow delivered to a patient at a patient interface during a synchronized high-flow mode, the method comprising:
delivering a constant baseline flow of breathing gases at a flow valve upstream from a patient interface;
measuring an upstream flow and an upstream pressure, wherein the upstream flow and upstream pressure are determined at a position between the flow valve and the patient interface;
based on the upstream flow and the upstream pressure, estimating a time-varying interface pressure;
determining that the time-varying interface pressure at the patient interface is less than a baseline pressure;
based on the time-varying interface pressure and the baseline pressure, determining a time-varying delta flow; and
delivering, at the flow valve, the time-varying delta flow in addition to the constant baseline flow.

11. The method of claim 10, wherein the patient interface is a nasal cannula and the time-varying interface pressure is determined at nares of the nasal cannula.

12. The method of claim 10, wherein the method further comprises determining a resistance for a breathing circuit including the patient interface.

13. The method of claim 12, wherein estimating the time-varying interface pressure is further based on the resistance.

14. The method of claim 12, wherein the resistance is determined according to a calibration procedure that varies the upstream flow and the upstream pressure.

15. The method of claim 10, wherein the time-varying delta flow is further based on a pressure difference between the time-varying interface pressure and the baseline pressure.

16. The method of claim 15, wherein the time-varying delta flow is equal to zero, when the pressure difference is less than a threshold.

17. A ventilator capable of synchronizing flow delivered to a patient at a patient interface, the ventilator comprising:
a flow valve fluidly coupled to a breathing circuit including a patient interface;
a flow sensor positioned downstream from the flow valve and upstream from the patient interface;
a pressure sensor positioned downstream from the flow valve and upstream from the patient interface;
a processor;
memory storing instructions that, when executed by the processor, cause the ventilator to perform a set of operations comprising:
determining a resistance for the breathing circuit including the patient interface;
delivering a baseline flow at the flow valve;
measuring an upstream flow at the flow sensor and an upstream pressure at the pressure sensor;
based on the upstream flow and the upstream pressure, estimating an interface pressure at the patient interface;
based on the interface pressure, determining a delta flow; and
delivering an augmented flow through the patient interface, wherein the augmented flow equals the baseline flow plus the delta flow.

18. The ventilator of claim 17, wherein the delta flow is proportional to a pressure difference between the interface pressure and the baseline pressure when the pressure difference is at least a threshold.

19. The ventilator of claim 17, wherein the delta flow is time-varying and non-negative.

20. The ventilator of claim 17, wherein determining the resistance includes performing a calibration procedure by varying the upstream flow and the upstream pressure.
